(19) United States
(12) Patent Application Publication
(10) Pub. No.: US 2010/0132721 A1
(43) Pub. Date: Jun. 3, 2010

(54) RESPIRATOR HELMET WITH INTEGRATED HEARING PROTECTION

(75) Inventor: Garth Graham Ivory, Christchurch (NZ)

Correspondence Address: BLAKELY SOKOLOFF TAYLOR & ZAFMAN LLP, 1279 OAKMEAD PARKWAY, SUNNYVALE, CA, 94085-4040, US

(73) Assignee: RPB, Ltd., Los Angeles, CA (US)

(21) Appl. No.: 12/326,771

(22) Filed: Dec. 2, 2008

(51) Int. Cl. A61F 11/00; A62B 18/00; A42B 3/00; A61F 11/06

(52) U.S. Cl. 128/864; 128/201.23; 2/410; 381/71.6

(57) ABSTRACT

An apparatus including a housing forming a protective head cover and a hearing protection mechanism coupled to the housing is defined. The apparatus further includes an attachment mechanism coupled to the housing and the attachment mechanism is to connect the housing to an external air supply.

### BACKGROUND

1. Field of the Invention

Embodiments of the present invention relate to a protective head cover. Specifically, the embodiments of the present invention relate to a helmet including a hearing protection mechanism and an attachment mechanism to connect to an external air supply.

2. Description of the Related Art

Protective head covers, for example helmets, are worn to protect the head of the wearer in a wide variety of environments. Some of these environments include construction sites, steel fabrication sites, foundries, mines and military bases. Construction workers, steelworkers, miners and soldiers wear helmets to protect their heads from being hit by falling debris or construction material or objects propelled by abrasive blasting equipment, explosives or ordinance. In some of these environments, it is also necessary for the wearers of helmets to utilize respirators to protect themselves from breathing in dust, harmful particulate matter or noxious gases.

In these environments, it is also desirable to prevent damage to the auditory senses that can be caused by explosions, construction equipment, power tools or abrasive blasting equipment. This environmental noise can be generated at high decibel levels as well as at very high frequencies. Earplugs and earmuffs are used to protect the auditory senses in these environments. Use of earplugs and earmuffs can be uncomfortable in connection with the wearing of a helmet. Also, they can render the wearer unable to receive audio communications. As a result, communications require the removal of hearing protection and sometimes the disabling of the noise generating equipment thereby reducing worker productivity.

### BRIEF DESCRIPTION OF THE DRAWINGS

The invention is illustrated by way of example and not by way of limitation in the figures of the accompanying drawings in which like references indicate similar elements. It should be noted that references to “an” or “one” embodiment in this disclosure are not necessarily to the same embodiment, and such references mean at least one.

FIG. 1 is a diagram of an exterior view of one embodiment of a hearing protection mechanism, an adjustable strap and a liner insert.

FIG. 2 is a diagram of an exterior view of one embodiment of a protective head cover and a hearing protection mechanism decoupled from the protective head cover.

FIG. 3A is a diagram of an exterior view of one embodiment of a hearing protection mechanism.

FIG. 3B is a diagram of an exterior view of one embodiment of a hearing protection mechanism.

FIG. 3C is a diagram of a side view of one embodiment of a hearing protection mechanism.

FIG. 4 is a diagram of an exterior view of one embodiment of a hearing protection mechanism.

FIG. 5 is a diagram of an exterior view of one embodiment of a protective head cover, a hearing protection mechanism and an attachment mechanism.

### DETAILED DESCRIPTION

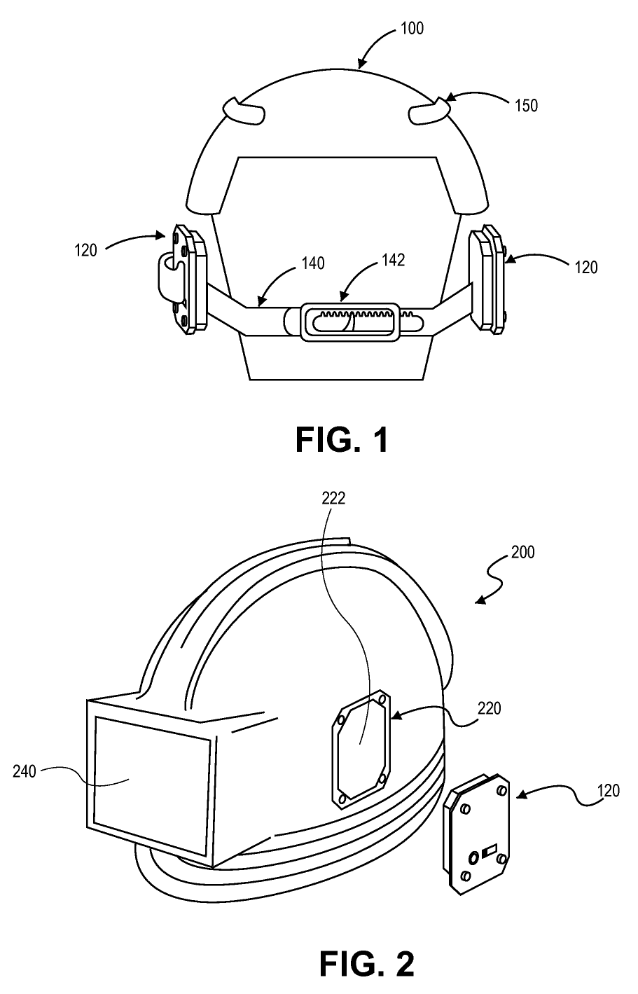

FIG. 1 is a diagram of an exterior view of one embodiment of a hearing protection mechanism, an adjustable strap and a liner insert. A liner insert 100 is coupled to or incorporates an adjustable strap 140. A set of hearing protection mechanisms 120 is attached to the adjustable strap 140. A “set” as used herein, refers to any positive whole number of items including one item. The liner insert 100 and adjustable hearing protection mechanisms 120 provide a one size fits all solution for an add-on hearing protection system within protective head covers.

The liner insert 100 can be inserted into the interior space of a protective head cover. The hearing protection mechanism 120, coupled to the liner insert 100 by the adjustable strap 140, protects the auditory senses of the wearer of the protective head cover from high-frequency and high decibel sounds. In one embodiment, the hearing protection mechanism 120 can also enable the wearer to hear sound and music through a speaker system embedded within the hearing protection mechanism 120.

The liner insert 100 is capable of conforming to the shape and size of the head of the wearer of the protective head cover so that it provides a better fit to the wearer. In one example embodiment, the liner insert 100 has a generally dome-like shape with a height between one to three inches and/or depth or diameter between five to seven inches. In another embodiment, the liner insert 100 has a shape that covers the back portion of the head of the wearer of the protective head cover 200. The liner insert 100 can be formed wholly or partially of flexible plastics, foam materials or similar materials that are capable of absorbing energy and impacts, including, but not limited to expanded polystyrene, urethane foam and thermo plastic rubber. The liner insert 100 has a shape that is also complementary to the interior surface and space of the protective head cover. In one embodiment, the surface of the liner insert 100 includes fitting members 150. The fitting members 150 provide a form fit with the interior of the protective head cover. In another embodiment, the insert liner 100 is attached to the interior of the protective head cover by fasteners, adhesives or similar attachment mechanisms.

The adjustable strap 140 is coupled to the liner insert 100 and attaches to the set of hearing protection mechanisms 120 to enable the wearer of the protective head cover to adjust the position of the hearing protection mechanism 120 relative to the ear by lengthening or shortening the length of the adjustable strap 140. The adjustable strap 140 can employ any adjusting mechanism that adjusts the length of a strap, for example a ratchet and pawl. In one example embodiment, the adjustable strap defines a perforated or ratcheted portion 142 to enable incremental adjustment of the length of the adjustable strap 140. The length of the adjustable strap 140 can be adjusted to conform to the size of the head of the wearer. In one example embodiment, the adjustable strap 140 includes two straps connected at an adjusting mechanism and the length can be between ten to fifteen inches when adjusted. The width of the adjustable strap can be any size and may not be uniform. This allows the adjustable strap 140 to be adjusted to any user as a one size fits all structure. Similarly, adjusting the strap allows the hearing protection mechanism 120 to be properly positioned over the ears of any wearer. In one example embodiment, the width of the adjustable strap can be between 0.4 to 2 inches. In another embodiment, the adjustable strap 140 does not have an adjusting mechanism and, instead, the adjustable strap 140 is a U-shaped plastic or metal strap capable of pushing the hearing protection mechanisms attached to it towards the ears of the wearer of the protective head cover.

The adjustable strap 140 can be removably coupled to the liner insert 100 or can be integrally formed with the padding of the liner insert 100. The adjustable strap 140 is attached to a set of hearing protection mechanisms 120 by looping around a cross-bar, a swivel adjustment mechanism, a set of screws, nuts and bolts, adhesives or similar fastening devices. In one example embodiment, the adjustable strap 140 is attached to a set of hearing protection mechanism 120 by latchets that allow the angle of the hearing protection mechanism 120 to be adjustable to the shape of the head and ear position on the head. The adjustable strap can be formed wholly or partially of materials including, but not limited to, plastics (e.g., polyethylene, polycarbonate, polypropylene, polystyrene, or acrylonitrile butadiene styrene), metals (e.g., aluminum, steel, tin, titanium, or chrome), composite compounds (e.g., carbon fiber or fiberglass), and other resilient and lightweight materials.

FIG. 2 is a diagram of an exterior view of one embodiment of a protective head cover and a hearing protection mechanism. The protective head cover 200 is configured to secure a lens 240 and a hearing protection mechanism 120. The lens 240 and the hearing protection mechanism 120 are secured to the protective head cover 200.

The protective head cover 200, with the hearing protection mechanism 120 coupled to it, shields the head of the wearer of the protective head cover 200 against harm from falling or flying objects. The protective head cover 200 also protects the auditory senses of the wearer from high-frequency and high decibel sounds. In one embodiment, the protective head cover 200 can also enable the wearer to hear sound and music through the hearing protection mechanism 120.

The protective head cover 200 can be formed wholly or partially of materials including, but not limited to, plastics (e.g., polyethylene, polycarbonate, polypropylene, polystyrene, or acrylonitrile butadiene styrene), metals (e.g., aluminum, steel, tin, titanium, or chrome), composite compounds (e.g., carbon fiber or fiberglass), and other resilient and lightweight materials. The protective head cover 200 can have any size and shape to form a compartment to receive a human head. In one embodiment, the protective head cover 200 has a roughly cylindrical shape with a closed top and open bottom, with a height between ten and fifteen inches and width and/or depth or diameter between six to eight inches. In one embodiment, the protective head cover 200 covers the head of the wearer completely. In another embodiment, the protective head cover 200 partially covers the head of the wearer such that portions of the head are exposed.

The lens 240 is coupled to the protective head cover 200 and protects the eyes of the wearer of the protective head cover 200. The lens 240 can be formed of materials including, but not limited to, shatter-resistant plastics (e.g., polycarbonate) and similar materials of various impact resistance suitable for construction of a safety lens. The lens 240 can be polarized to protect the eyes of the wearer from the ultraviolet lights or other harmful radiations or energy sources. The lens 240 can have any size and shape complementary to the shape and size of an opening defined by the shape and size of the protective head cover 200. In one embodiment, the lens 240 has a roughly rectangular shape with a width between eight and ten inches, a height between three and six inches and a thickness between 0.04 and 0.2 inches at the thinnest point. The lens 240 can be flat or curved, depending on the shape of the protective cover 200.

The hearing protection mechanism 120 is coupled to the protective head cover 200. In one embodiment, the hearing protection mechanism 120 protrudes through an opening defined by the protective head cover 200. In another embodiment, the hearing protection mechanism 120 is coupled to an interior surface of the protective head cover 200 so that the hearing protection mechanism 120 is not externally visible. In another embodiment, the hearing protection mechanism 120 can be integrated into the protective head cover 200 and may not be detachable from the protective head cover 200. The hearing protection mechanism 120 is positioned to be adjacent to an ear of the wearer of the protective head cover 200. The hearing protection mechanism 120 protects the auditory senses of the wearer of the protective head cover 200. Further details of the hearing protection mechanism 120 are described below.

The protective head cover 200 includes a frame 220. The hearing protection mechanism 120 can be coupled to frame 220. The frame 220 is keyed to the hearing protection mechanism 120. As used hereinafter, keying refers to a fit such that the frame 220 and the hearing protection mechanism 120 are shaped complementary to each other so that only an object that has the exact size and shape of the hearing protection mechanism 120 fits into the frame 220. Likewise, the hearing protection mechanism 120 can be coupled to any protective head cover which includes a frame keyed to the hearing protection mechanism 120. This enables an older model of a hearing protection mechanism coupled to a protective head cover to be replaced with a newer hearing protection mechanism that provides better hearing protection. In one embodiment, the frame 220 is not keyed to the hearing protection mechanism 120 and defines a general space to receive and secure the hearing protection mechanism 120.

In one example embodiment, the frame 220 has a generally rectangular shape. The frame 220 defines a generally rectangular slot 222. The generally rectangular slot 222 has a height between four and five inches and a width between 2.5 and three inches so that the hearing protection mechanism 120 to which the frame 220 is keyed is comparable to a size of a human ear. The hearing protection mechanism 120 can be attached to the frame 220 by a set of screws or nuts and bolts or similar fastening devices.

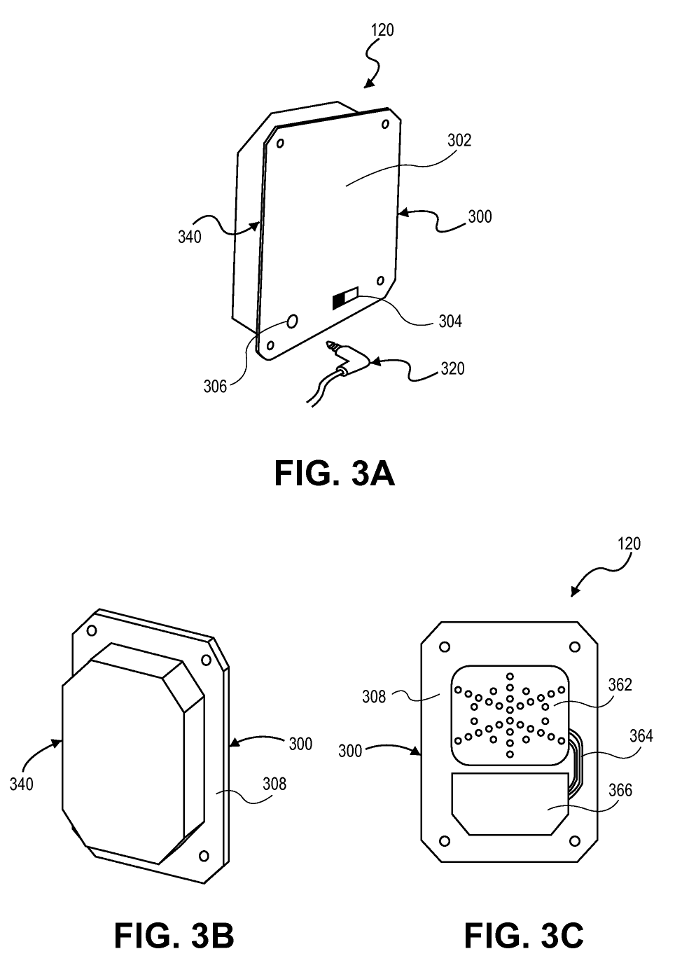

FIG. 3A is a diagram of an exterior view of one embodiment of a hearing protection mechanism. The hearing protection mechanism 120 includes a mounting mechanism 300 and a padding module 340. The mounting mechanism 300 couples the hearing protection mechanism 120 to a protective head cover by engaging the housing of the protective head cover. In one embodiment, the mounting mechanism 300 provides a substrate to which the padding module 340 can be coupled. In another embodiment, the mounting mechanism 300 forms a shell that wholly or partially encloses the padding module 340.

The mounting mechanism 300 can have a shape complementary to the shape of the housing of the protective head cover. In one example embodiment, the mounting mechanism 300 is a thin panel with a generally rectangular shape. The mounting mechanism 300 has a height between three and five inches and a width between 2.5 and four inches. The mounting mechanism 300 can be as thin as 0.04 inches or it can have the thickness of a protective head cover that the mounting mechanism 300 is received by. The mounting mechanism 300 can be formed wholly or partially of materials including, but not limited to, plastics (e.g., polyethylene, polycarbonate, polypropylene, polystyrene, or acrylonitrile butadiene styrene), metals (e.g., aluminum, steel, tin, titanium, or chrome), composite compounds (e.g., carbon fiber or fiberglass), and other resilient and lightweight materials.

The mounting mechanism 300 has an outer surface 302. A port 306 can be formed through the outer surface 302 to receive an audio signal from an audio source. The audio source could be a music player such as an MP3 player, or a communication device such as a mobile phone or a two-way radio. In one embodiment, a stereo audio jack 320 connected to the audio source can be plugged into the port 306 to transfer an audio signal from the audio source into the hearing protection mechanism 120. In another embodiment, the port 306 can be capable of receiving other kinds of signals, for example, a digital data signal. In another embodiment, a set of ports can be formed to receive different kinds of audio plugs and signals. An audio signal coming into the port 306 can be ready to go to an output device (i.e. a speaker) or it can be a signal to be further processed. The further processing of an audio signal, if necessary, is done by an audio-processing circuitry in the padding module 340. The port 306 is located near the lower left corner of the outer surface 302, but it can be located anywhere on the hearing protection mechanism 120.

An activation mechanism 304 can be attached to or protrude from the outer surface 302 to allow activation of the hearing protection mechanism 120. The activation mechanism 304 can be any type of switch or similar mechanism for powering on or off an electronic device. In one example embodiment, the activation mechanism 304 is a simple positional on-off switch. In another example embodiment, the activation mechanism 304 can be an on-off push button. The activation mechanism 304 is located near the lower right corner of the outer surface 302, but it can be located anywhere on the hearing protection mechanism 120. The activation mechanism 304 can have any size within the dimensions of the hearing protection mechanism 120. In one example embodiment, the activation mechanism 304 is generally rectangular with a height between 0.12 and 0.2 inches and a width about 0.4 inches.

FIG. 3B is a diagram of an exterior view of one embodiment of a hearing protection mechanism. The padding module 340 is coupled to the inner surface 308 of the mounting mechanism 300 so that the padding module 340 faces the wearer of the protective head cover 200 when the mounting mechanism 300 is coupled to the protective head cover 200. The padding module 340 can have any shape as long as it can be fitted within the edges of the mounting mechanism 300 and cover or form a perimeter around the ears the wearer. In one example embodiment, the padding module 340 is a generally rectangular pad with a height at least 0.25 inches smaller than that of the mounting mechanism 300 and a width at least 0.25 inches smaller than that of the mounting mechanism 300 so that it fits within the edges of the mounting mechanism 300. The thickness of the padding module 340 depends on the thickness of the mounting mechanism 300 and the thickness of the protective head cover 200 that the mounting mechanism 300 to be coupled to. The sum of the thicknesses of padding module 340 and the mounting mechanism 300 will generally correspond to the thickness of the protective head cover 200. In one example embodiment, the padding module 340 has a thickness between 0.4 and 2 inches. The padding module 340 is coupled to the inner surface 308 of the mounting mechanism 300 by a set of screws or nuts and bolts or similar fastening devices or adhesives. The padding module can be formed wholly or partially of materials including, but not limited to open cell and closed cell foam, thermoplastic rubber, urethane and flexible plastics.

The padding module 340 can be formed from materials, for example memory foam, that are capable of isolating the ambient noise from an ear of the wearer of a protective head cover. In one embodiment, the padding module 340 is entirely a memory foam block, which can be covered with a fabric cover.

FIG. 3C is a diagram of a side view of one embodiment of a hearing protection mechanism. The hearing protection mechanism 120 includes a speaker mechanism 362, an audio processing circuitry 366 and a set of wires 364 that connects the speaker mechanism 362 and the audio processing circuitry 366. The speaker mechanism 362 and the audio processing circuitry 366 can be integrated into one unit. In one embodiment, the speaker mechanism 362, the audio processing circuitry 366 and the set of wires 364 are attached to the mounting mechanism 300 by a set of screws or nuts and bolts or similar fastening devices or adhesives. In another embodiment, the speaker mechanism 362 and the audio processing circuitry are covered by the padding module 340 that is coupled to the mounting mechanism 462. In another embodiment, the speaker mechanism 362 and the audio processing circuitry 366 can be defined within the padding module 340.

The speaker mechanism 362 and the audio processing circuitry 366 can have any shape that fits within the edges of the mounting mechanism 300. In one example embodiment, the speaker mechanism 362 and the audio processing circuitry 366 are placed adjacent to each other and they form a generally rectangular shape with a height at least 0.25 smaller than that of the mounting mechanism 300 and a width at least 0.25 and one inches smaller than the width of the mounting mechanism 300 so that they fit within the edges of the mounting mechanism 300. In another embodiment, the speaker mechanism 362 wholly or partially covers the audio processing circuitry 366 so that the speaker mechanism 362 is placed closer to the ear of the wearer of the protective head cover than the processing circuitry 366 is. The thickness of the speaker mechanism 362 and the audio processing circuitry 366 depends on the thickness of the mounting mechanism 300 and the thickness of a protective head cover to which the mounting mechanism 300 is to be coupled. The sum of the thicknesses of padding module 340 and the mounting mechanism 300 will generally correspond to the thickness of the protective head cover. In one example embodiment, the speaker mechanism 362 and the audio processing circuitry 366 have a thickness between 0.4 and two inches.

The speaker mechanism 362 can output audio signal from an audio source, for example, audio signals received through the port 306. The audio processing circuitry 366 includes an active noise cancelling mechanism that includes an audio sensor and a circuitry that dampens the noise detected by the sensor. In one embodiment, the audio processing circuitry 366 can process the audio signals received through the port 306 on the hearing protection mechanism 120 and send the processed signals to the speaker mechanism 362 through the set of wires 364. In another embodiment, the audio processing circuitry 366 includes or is connected to a receiver, for example, a Bluetooth™ receiver, which is capable of receiving an audio signal wirelessly from an external audio source.

In one embodiment, the active noise cancelling mechanism has a noise cancellation range of up to 8,000 Hz, which is sufficient to cover 6,000-8,000 Hz blasts. In another embodiment, the active noise cancelling mechanism has a noise cancellation range of up to 10,000 Hz. In one embodiment, the hearing protection mechanism 120, with the active noise cancelling mechanism and a noise isolation mechanism such as a memory foam block, provides up to 30 db noise reduction. In another embodiment, the hearing protection mechanism 120 provides up to 40 db noise reduction. The hearing protection mechanism 120 can include a battery to supply power to the audio processing circuitry 366 and to the speaker mechanism 362. In another embodiment, the hearing protection mechanism 120 can be connected to an external power supply.

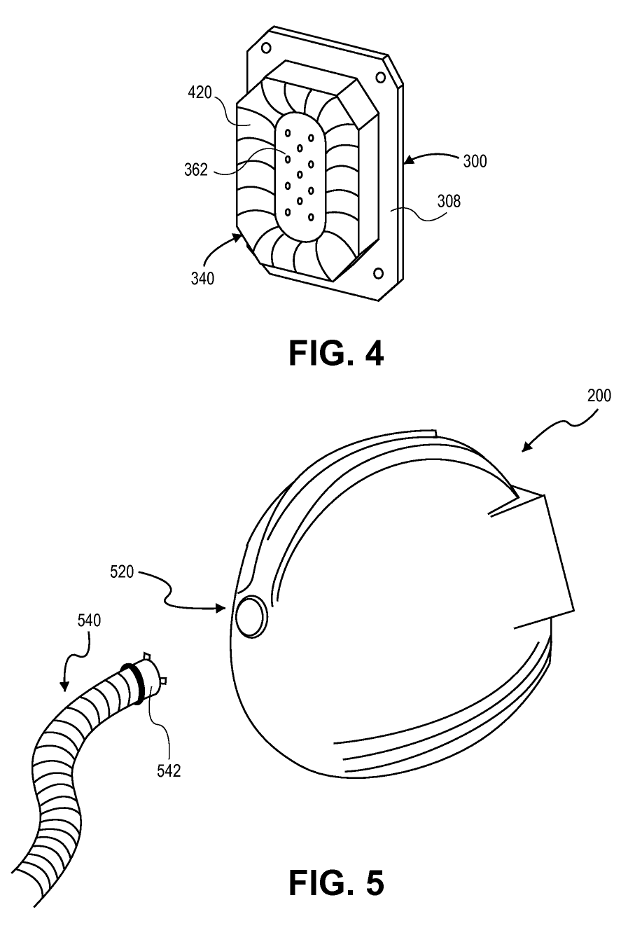

FIG. 4 is a diagram of an exterior view of one embodiment of a hearing protection mechanism. The padding module 340 is coupled to the inner surface 308 of the mounting mechanism 300. In one embodiment, the padding module 340 includes the speaker mechanism 362, the audio processing circuitry as described above and a memory foam member 420. The audio processing circuitry is positioned between the speaker mechanism 362 and the mounting mechanism 300 so that it is not externally visible. The memory foam member 420 can form a perimeter around the ear of the wearer of the protective head cover to isolate the ambient noise from the ear. The speaker mechanism 362 is exposed to the ear of the wearer through the opening defined by the memory form member 420. The memory foam member 420 can be formed of memory foam, polyurethane foam or similar noise absorbing materials.

FIG. 5 is a diagram of an exterior view of one embodiment of a protective head cover, a hearing protection mechanism and an attachment mechanism. An attachment mechanism 520 is coupled to the protective head cover 200 to connect the protective head cover to an external air supply. A hose 540 is connected to an external air supply. In one example embodiment, the attachment mechanism 520 includes a circular slot that receives an end portion 542 of the hose 540 to supply air into the protective head cover 200 from the external air supply. In another embodiment, the protective head cover 200 incorporates an internal air supply. An air supply can be an air filtration system, respirator system, ventilation system or similar air supply source that is mounted externally or internally.

In the foregoing specification, the invention has been described with reference to specific embodiments thereof. It will, however, be evident that various modifications and changes can be made thereto without departing from the broader spirit and scope of the invention as set forth in the appended claims. The specification and drawings are, accordingly, to be regarded in an illustrative rather than a restrictive sense.

What is claimed is:

1. An apparatus comprising:
a housing forming a protective head cover;
a hearing protection mechanism coupled to the housing; and
an attachment mechanism coupled to the housing, the attachment mechanism to connect the housing to an external air supply.

2. The apparatus of claim 1, further comprising:
an adjustment mechanism to adjust a placement of the hearing protection mechanism within the protective head cover.

3. The apparatus of claim 1, wherein the protective head cover defines an inbound space to receive positive atmospheric pressure from the external air supply.

4. The apparatus of claim 1, wherein the hearing protection mechanism comprises:
a noise isolation mechanism.

5. The apparatus of claim 1, wherein the hearing protection mechanism comprises:
an active noise cancelling mechanism.

6. The apparatus of claim 1, wherein the hearing protection mechanism comprises:
a speaker mechanism to output audio signals from an audio source.

7. The apparatus of claim 1, wherein the hearing protection mechanism comprises:
a port through which to receive an audio signal from an external source.

8. The apparatus of claim 1, wherein the hearing protection mechanism comprises:
a receiver to receive an audio signal wirelessly from an audio source.

9. The apparatus of claim 1, wherein the hearing protection mechanism has a noise cancellation range of up to 8,000 Hz.

10. The apparatus of claim 1, wherein the hearing protection mechanism comprises:
a foam member to cover an ear.

11. The apparatus of claim 11, wherein the hearing protection mechanism comprises:
an activation mechanism to activate the hearing protection mechanism.

12. An apparatus comprising:
a mounting mechanism to attach the apparatus to a protective head cover, the protective head cover coupled to an external air supply; and
a padding module coupled to the mounting mechanism, the padding module to dampen a sound level within the protective head cover.

13. The apparatus of claim 12, further comprising:
an adjustment mechanism to adjust a placement of the hearing protection mechanism within the protective head cover.

14. The apparatus of claim 12, wherein the protective head cover defines an inbound space to receive positive atmospheric pressure from the external air supply.

15. The apparatus of claim 12, wherein the padding module comprises:
a noise isolation mechanism.

16. The apparatus of claim 12, wherein the padding module comprises:
an active noise cancelling mechanism.

17. The apparatus of claim 12, wherein the padding module comprises:
a speaker mechanism to output audio signals from an audio source.

18. The apparatus of claim 12, wherein the padding module comprises:
a port through which to receive an audio signal from an external source.

19. The apparatus of claim 12, wherein the padding module has a noise cancellation range of up to 8,000 Hz.

20. The apparatus of claim 12, wherein the padding module comprises:
a foam member to cover an ear.
